(19) United States
(12) Patent Application Publication
(10) Pub. No.: US 2001/0042371 A1
(43) Pub. Date: Nov. 22, 2001

(54) Programmable open and closed loop electronics for control of an internal combustion engine

(76) Inventors: Corinna Topfer-Hartung, Bad Honnef (DE); Karl Stellwagen, Frankenthal (DE); Boris Besler, Karlsruhe (DE)

Correspondence Address: Charles L. Schwab, NEXSEN PRUET JACOBS & POLLARD, LLC, Hardaway/Mann IP Group, P.O.Box 10107, Greenville, SC, 29603, US

(21) Appl. No.: 09/770,826

(22) Filed: Jun. 11, 2001

(30) Foreign Application Priority Data
Feb. 1, 2000 (DE) 100 04 330.5

(51) Int. Cl. F01N 3/00; F02D 41/14; G01N 33/497

(52) U.S. Cl. 60/276; 123/679; 73/23.32

(57) ABSTRACT

Programmable open and closed loop electronics for control of an internal combustion engine.

The following functions are integrated in a known method and the corresponding device: “closed loop combustion control of a gas engine, program for automatic starting and stopping of the unit, monitoring of the temperatures and pressures of gas, water, lubricant and exhaust gas and of levels inside the installation, driving of the ignition unit, setting of the speed controller, control of the auxiliary circulation systems, monitoring of the generator winding temperature, self-monitoring of the control unit and of the probes, and storage of the measured values for monitoring and diagnostic purposes.”

According to the invention, the concentration of nitrogen oxides (NOₓ) and/or of fluorine and/or of chlorine in the exhaust gas of the internal combustion engine is quantitatively measured with an exhaust gas sensor (6) continuously while the internal combustion engine is operating. The values of the nitrogen oxide emission are especially suitable both for monitoring and also for open and closed loop control of an appropriately equipped internal combustion engine.

This invention relates to a method for open and closed loop control and monitoring of an internal combustion engine with programmable open and closed loop control electronics to which measurement signals from sensors and/or transmitters arranged on the internal combustion engine are supplied, the open and closed loop control electronics driving control elements and/or actuators for intervention in the operation of the internal combustion engine, and the open and closed loop control electronics exhibiting an interface for data interchange, as well as appropriately designed open and closed loop control electronics.

Such a method and a corresponding device is known from the DEUTZ MWM company publication “TEM System.” The following functions are integrated into this TEM System: “closed loop combustion control of a gas engine, program for automatic starting and stopping of the unit, monitoring of the temperatures and pressures of gas, water, lubricant and exhaust gas and of levels inside the installation, driving of the ignition unit, setting of the speed controller, control of the auxiliary circulation systems, monitoring of the generator winding temperature, self-monitoring of the control unit and of the probes, and storage of the measured values for monitoring and diagnostic purposes.” The TEM System is capable of performing all these functions in intelligent fashion and thus permits especially reliable running of the internal combustion engine or installation under all service conditions.

It is an object of the invention to improve the previously described system and method.

According to the invention this object is achieved by virtue of the fact that the concentration of nitrogen oxides (NOₓ) and/or of fluorine and/or of chlorine in the exhaust gas of the internal combustion engine is quantitatively measured with an exhaust gas sensor continuously while the internal combustion engine is operating. The values of, in particular, the nitrogen oxide emission are especially suitable both for monitoring and also for open and closed loop control of an appropriately equipped internal combustion engine. Thus these values, referred to a particular operating point of the internal combustion engine, defined for example by measurement of the speed of the quantity of fuel delivered and, if applicable, of further operating parameters such as various temperatures, convey information about the quality of the proceeding combustion of fuel and combustion air and inferences to be derived therefrom. This information can be stored in appropriate storage components of the open and closed loop control electronics for later interpretation or also, however, processed for, in particular, the active open and closed loop control of the internal combustion engine. Intervention in the running of the motor is also necessary if substances harmful for the operation of the installation (fluorine, chlorine) are-measured. These substances occur in some instances when operating on landfill gas. In this case, an exhaust gas sensor for measuring the concentration of fluorine and/or an exhaust gas sensor for measuring the concentration of chlorine is employed in addition to the exhaust gas sensor for measuring the concentration of nitrogen oxides. The exhaust gas sensor for measuring the concentration of nitrogen oxides is thus always normally or preferably present.

In development of the invention, the exhaust gas sensor exhibits an ionically conductive, in particular a cationically conductive, ceramic (solid electrolyte), an auxiliary electrode for adaptation to the type of gas, a reference electrode and a working electrode. With an exhaust gas sensor designed in this fashion it is possible, without further equipment cost, to measure continuously the concentration of the nitrogen oxide emission of an internal combustion engine while it is operating continuously. This simple structure of the exhaust gas sensor constitutes the essential distinction to known nitrogen oxide analysis devices, which were suitable only for discontinuous measurements because of their high equipment cost and the continuing necessity of calibration. An example of such an analysis method is a measurement principle that utilizes chemoluminescence (emission of light induced by a chemical reaction) in the band between 590 and 3000 nm when nitrogen monoxide (NO) reacts with ozone (O₃).

In further development of the invention, an electromotive force (EMF) (of freely mobile electrons) between the reference electrode and the auxiliary electrode of the exhaust gas sensor is evaluated for an output voltage signal directly proportional to the gas concentration. In further development, the gas concentration is then measured potentiometrically. This exhaust gas sensor can be immediately adapted to other gases for measurement (for example fluorine, chlorine) by modification of the auxiliary electrode. Operation with landfill gases can be made reliable by measuring chlorine or fluorine. If such constituents occur in the exhaust gas and if they are detected by the selective sensor, the motor can be, for example, shut off by the control unit according to the invention in order to avert damage.

In development of the invention, the open and closed loop control electronics so designed is suitable, in particular, for driving the actuator of a mixture controller of a gas motor, in particular an Otto-cycle gas motor. Here a nonoptimally adjusted gas/air mixture directly affects the concentration of nitrogen oxide. The invention is also advantageously applicable in the case of compression-ignition internal combustion engines; here the beginning of delivery and, if applicable, also the splitting of injection into a preinjection and a main injection is then adjusted on the basis of the measured and interpreted values or concentrations.

In further development of the invention, the open and closed loop control electronics is equipped with further components as required. Thus there is, for example, a “running smoothness measurement” component, with which the torque delivered by the internal combustion engine to the crankshaft is determined in a cylinder-selective fashion. This device makes it possible, in particular, to detect defects in combustion that can be precisely assigned in a cylinder-selective fashion, and appropriate corrective steps can be put into effect. Thus, for example, it can be established whether the energy delivered to a spark plug of a cylinder in a gas motor is adequate for reliable combustion of the mixture present in the combustion chamber or, however, a greater ignition energy must be delivered, for example as a consequence of aging or wear of the spark plug. This was previously possible only by measuring the temperature in every combustion chamber of the internal combustion engine.

Further advantageous developments of the invention can be inferred from the description of the drawings, in which an exemplary embodiment of the invention illustrated in the Figures is described more thoroughly.

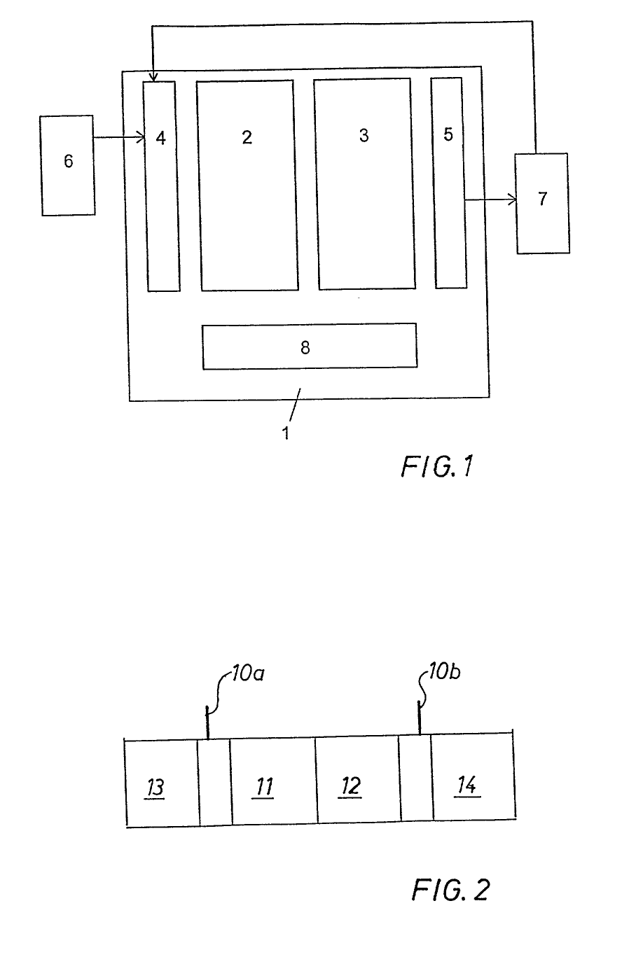

FIG. 1 shows a block diagram of the programmable open and closed loop control electronics.

FIG. 2 shows a schematic diagram of the NOₓ sensor.

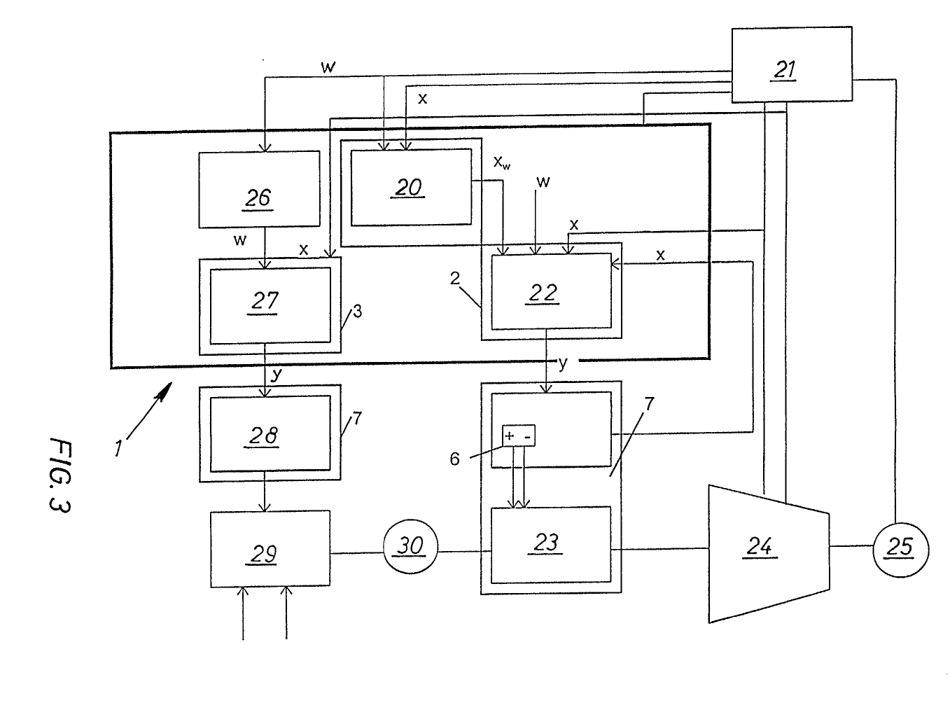

FIG. 3 shows closed loop control circuits of the programmable open and closed loop control electronics.

The block diagram of FIG. 1 shows programmable open and closed loop control electronics 1, the GEM, which is an automatically operating measurement and open and closed loop control device for sensor and actuator management. It is usable for all kinds of internal combustion engines, in particular, however, compression-ignition internal combustion engines and gas motors based on-Otto-cycle motors or diesel motors. With this device, several dependent or linked tasks can be performed, and the device is designed specially for motors that must function reliably under harsh environmental conditions and at service temperatures from −15° C. to +80° C. The device or system is designed to conform to the appropriate protective classes, and thus all inputs and outputs are protected against overvoltages and short circuits and are diagnosable, that is, the software can determine whether a short circuit or an open circuit is present (plausibility test). What is more, a self-diagnosis of the internal hardware in this respect is carried out. Control valves can be driven directly via a pulse-width modulated (PWM) signal or via integrated drive electronics.

The device is built into a sturdy die-cast aluminum housing and thus offers both immunity to electromagnetic interference and reliable protection against mechanical stresses. Programmable open and closed loop control electronics 1 includes essentially the functional modules “motor control” with speed control, antiknock control and ignition system and “motor monitoring” with emission control, running smoothness monitoring and sensors 6. Sensors 6 mounted on the internal combustion engine supply all relevant physical values to the electronics. Programmable open and closed loop control electronics 1 controls and monitors the motors in accordance with this information about the instantaneous motor condition and the setpoints/operating data of the installation management. In case of values greater than or less than the specified limits, actions are initiated that safeguard the motor through warnings and/or shutdowns when abnormal operating conditions occur. These violations of the specified operating parameters are stored in an operating log and thus can be used as required for damage diagnosis (readout of the malfunction memory for troubleshooting, for visualization, and for servicing or customer service purposes). The mechanical data are recorded in paperless electronic fashion. An appropriately specified CAN port is also present in order to permit data interchange with other intelligent units and systems (installation management, that is, higher-level control/control facility/service laptop). This drastically reduces the cost of wiring, which otherwise is usually very great. Characteristic curves, parameters and calibration data for sensor and actuator management as well as control configurations can be stored in a nonvolatile memory. External access to these data is possible via editor software and a laptop.

Programmable open and closed loop control electronics 1 contains as central elements a control unit 2 and a monitoring unit 3. It further exhibits inputs 4 and outputs 5 and an interface 8 for data interchange. Inputs 4 can be connected to sensors/transmitters 6, and outputs 5 with control elements/actuators 7, these also simultaneously being connected to inputs 4. Control unit 2 contains processors for speed control, antiknock control and the ignition system. Monitoring unit 3 monitors and controls the mixture as well as the emissions. Further, running smoothness, which simultaneously represents detection of missing in cylinders, is monitored here. There are sensors 6 for motor coolant inlet temperature, motor coolant discharge temperature, receiver temperature, receiver pressure, lubricant pressure, vibration/body-borne noise, speed, pulse sensor, ignition timing, throttle valve position, No, measurement or fluorine and/or chlorine measurement, and, optionally, for lubricant temperature, lubricant quality and lubricant level. Alternatively to sensor 6 for the throttle valve position, there is, of course, a sensor 6 for the control rod position (if present) or other motor-specific elements when programmable open and closed loop control electronics 1 is used with a diesel motor.

The control units present for a gas motor are specified below.

1. Ignition

With the aid of a digital ignition unit as a component of control unit 2, which is microprocessor-controlled, the ignition point and the distribution of ignition energy for each individual cylinder are calculated. The compressed gas-air mixture is ignited at the spark plug by a high-voltage spark discharge generated by the ignition coil associated with the cylinder. The ignition point can be preadjusted in dependence on the kind of gas or the speed with allowance for the speed signal 6. The ignition point is adjusted in cylinder-selective fashion, and the ignition energy is 125 mJ to 150 mJ.

2. Mixture Control

Mixture control is effected in monitoring unit 3 for the adjustment of the optimal fuel-air ratio for all operating conditions. To this end, the concentration of NOₓ in the exhaust gas is measured with a sensor 6 (FIG. 2) and further processed. This information is used for driving control element 7, in the form of an actuator, from the mixture controller. In this way, the optimal fuel-air ratio is set and thus the desired pollutant values are maintained. Intervention in the running of the motor is likewise possible if harmful constituents (fluorine, chlorine) are present.

3. Knock Control

Knock control is again a component of control unit 2, and this is used in order to achieve optimal control for each individual cylinder in case of fluctuating gas quality with respect to the methane index or knock resistance of the mixture. The basis of this control is measurement of body-borne noise with electrical sensors 6. The signals are digitized and then further processed. In case of knocking combustion, the ignition point is altered and the ignition angle is changed in the “retard” direction. If the specified ignition angle is exceeded, a power reduction is effected. If the value goes below the limit again, the power can be increased again and the ignition point adjustment reversed. In case of very great reduction in the methane index, a power reduction is performed.

4. Detection of Missing in Cylinders

This is again a component of monitoring unit 3 and makes it possible to determine the torque delivered to the crankshaft in a cylinder-selective fashion on the basis of highly precise speed measurement 6 and determination of speed irregularity. In this way, it is possible to diagnose or assess defects in combustion or defects in compression.

5. Speed Control (Power Control)

This is again a component of control unit 2. In order to be able to hold the speed constant under changing load conditions, it is necessary to bring positioning motor 7 of the throttle valve into a desired position using a current regenerated in the programmable open and closed loop control electronics and thus exert open loop control over the motor speed or power. The quantity of fuel gas-air mixture delivered in the cylinder and the resultant torque is controlled via the opening of the throttle valve. Speed measurement involves detecting the pulses of crankshaft transmitter 6 and of camshaft transmitter 6 and thus, by performing a nominal-actual comparison, controlling the equilibrium of torque required and torque produced. Control is based on the power requirement or torque requirement of the driven machines.

NOₓ sensor or fluorine or chlorine sensor 6 exhibits two ports 10a, 10b, which are suitably connected to inputs 4. The NOₓ sensor proper exhibits a cationically conductive ceramic in the form of solid electrolyte 11, which is arranged next to an auxiliary electrode 12. Auxiliary electrode 12 can be adapted to each kind of gas through appropriate modification. There are, further, a reference electrode 13 and a working electrode 14. Sensor 6 utilizes the electromotive force (EMF) of freely mobile electrons between reference electrode 13 and auxiliary electrode 12, the electromotive force (EMF) generating an output voltage signal that is, in turn, directly proportional to the gas concentration. Thus the gas concentration is measured potentiometrically.

FIG. 3 shows the principal control circuits of programmable open and closed loop control electronics 1. Programmable open and closed loop control electronics 1 exhibits a power controller 20, to which a corresponding setpoint value w and an actual value x are supplied from a control plane 21. Power controller 20 emits a signal x_{w}, which is supplied to a speed controller 22, which is a component of control unit 2, along with the setpoint value w and the actual value x. Speed controller 22 emits a signal y with which a control element 7 is driven in order to adjust throttle valve 23. To this end, there is a further element that detects throttle valve control element position 23a. From internal combustion engine 24 and one driven generator 25 upon which open or closed loop control is exerted, respective operating parameters are acquired and supplied, via control plane 21 and inputs 4, to programmable open and closed loop control electronics 1. Further, the power characteristic curve 26 is stored in the programmable open and closed loop control electronics and is supplied to mixture controller 27, a component of monitoring unit 3. The mixture controller drives an actuator 28 in the form of a control element 7, which adjusts the ratio of air and gas supplied by a gas mixer 29. This mixture is supplied to internal combustion engine 24 via a turbocharger 30 and throttle valve 23.

1. Method for open and closed loop control and monitoring of an internal combustion engine with programmable open and closed loop control electronics to which measurement signals from sensors and/or transmitters arranged on the internal combustion engine are supplied, the open and closed loop control electronics driving control elements and/or actuators for intervention in the operation of the internal combustion engine, and the open and closed loop control electronics exhibiting an interface for data interchange,
characterized in that the concentration of nitrogen oxides (NOₓ) and/or of fluorine and/or of chlorine in the exhaust gas of the internal combustion engine is quantitatively measured with an exhaust gas sensor (6) continuously while the internal combustion engine is operating.

2. Method according to claim 1,
characterized in that the actuator (28) of a mixture controller (27) of a gas motor is driven by the control element and/or actuator (7).

3. Method according to one of the foregoing claims,
characterized in that the open and closed loop control electronics (1) exhibits a “running smoothness measurement” component, with which the torque delivered by the internal combustion engine to the crankshaft is determined in a cylinder-selective fashion.

4. Open and closed loop control electronics for the open and closed loop control and monitoring of an internal combustion engine, measurement signals from sensors and/or transmitters arranged on the internal combustion engine being supplied to the open and closed loop control electronics, the open and closed loop control electronics driving control elements and/or actuators for intervention in the operation of the internal combustion engine, and the open and closed loop control electronics exhibiting an interface for data interchange,
characterized in that there is an exhaust gas sensor (6), which continuously performs quantitative measurement of the concentration of nitrogen oxides (NOₓ) and/or of fluorine and/or of chlorine in the exhaust gas of the internal combustion engine while the internal combustion engine is operating.

5. Open and closed loop control electronics according to claim 4,
characterized in that the exhaust gas sensor (6) exhibits an ionically conductive, in particular cationically conductive, ceramic (solid electrolyte) (11), an auxiliary electrode (12) for adaptation to the kind of gas, a reference electrode (13) and a working electrode (14).

6. Open and closed loop control electronics according to one of the foregoing claims 4 or 5,
characterized in that an electromotive force (EMF) between reference electrode (13) and auxiliary electrode (12) of the exhaust gas sensor (6) is evaluated for an output voltage signal directly proportional to the gas concentration.

7. Open and closed loop control electronics according to one of claims 4 to 6,
characterized in that the gas concentration is measured potentiometrically.

8. Open and closed loop control electronics according to one of claims 4 to 7,
characterized in that the open and closed loop control electronics (1) exhibits a “running smoothness measurement” component, with which the torque delivered by the internal combustion engine to the crankshaft is determined in a cylinder-selective fashion.
